(19) United States
(12) Patent Application Publication
(10) Pub. No.: US 2008/0319389 A1
(43) Pub. Date: Dec. 25, 2008

(54) Medical Devices with Rigid Rod Paraphenylene

(76) Inventors: Patricia A. Davis-Lemessy, Miami Lakes, FL (US); Thomas Neil Trotta, Miami Shores, FL (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/663,696

(22) PCT Filed: Sep. 28, 2005
(86) PCT No.: PCT/US05/34937

§ 371 Date: Jun. 9, 2008

Related U.S. Application Data
(60) Provisional application No. 60/613,798, filed on Sep. 28, 2004.

(51) Int. Cl. A61M 25/10; A61M 25/00; A61M 25/09

(52) U.S. Cl. 604/103.06; 604/523; 604/528

(57) ABSTRACT

The medical devices of the present invention include self-reinforcing polymer materials, based on the rigid-rod paraphenylene copolymer chemistry. Accordingly, the materials of the present invention provide a unique combination of physical properties that are advantageous for use in various medical devices. The medical devices may include those for maneuvering through the body passages of a patient, such as for example the circulatory system, to a desired site for treatment.

### CROSS-REFERENCE To RELATED APPLICATION

This application claims the benefit of priority of U.S. Provisional Patent Application No. 60/613,798 filed on Sep. 28, 2004.

### BACKGROUND AND SUMMARY OF THE INVENTION

1. Technical Background

The present invention relates generally to medical devices, and more particularly to medical devices including rigid-rod paraphenylene polymer materials.

2. Discussion

Material selection can be important for medical devices. Many such medical devices are made from thermoplastic polymers. In many cases, the thermoplastic is reinforced with other materials to achieve the desired mechanical properties.

It would be desirable to provide a material having the desired mechanical properties without additional reinforcement. A family of polymers has been developed by Mississippi Polymer Technology, which are self-reinforcing polymers based on the rigid-rod paraphenylene copolymer chemistry. As a result, no additional material is needed for reinforcing purposes.

This family of high performance thermoplastics is available under the trademark Parmax®, which are high-temperature engineered polymers in the rigid-rod paraphenylene family. The glass transition temperature (Tg) is reported to be 155° C. Features of these polymers include high compression and flexural strengths without reinforcement, as well as chemical and wear resistance, noncombustibility, corrosion resistance, scratch resistance, and very low moisture absorption. In addition, the rigid-rod paraphenylene polymers exhibit high melt strength and high melt viscosity. Extrusion and compression molding temperature requirements are in the 200-300° C. range.

In addition, these polymers can be solvent-cast into thin films from various solvent mixtures, including 1-methly-2-pyrrolidone (NMP) or NMP-toluene, NMP-methylene chloride and NMP-chlorobenzene.

An example of medical devices that may be made of these polymers are catheter shafts. Stronger, thinner catheter shafts are desirable in various types of medical devices, including cardiovascular, endovascular and neurovascular catheter products. Self-reinforcing polymer shafts in the rigid-rod paraphenylene family of polymer materials provide superior shafts for these types of catheters.

The self-reinforcing nature of rigid-rod paraphenylene allows for thinner wall extrusions without sacrificing strength, which translates into catheter deliverability, pushability and steerability.

Catheters are often intended to follow a specific path through body passages selected by a physician. There are many different kinds and types of catheters, including for example balloon catheters, diagnostic catheters, guiding catheters, stent delivery system catheters, injection catheters, gene therapy catheters, electrophysiology catheters, therapeutic drug delivery catheters, ultrasound catheters, laser angioplasty catheters, etc.

Structurally, catheters may have a flexible shaft extending between a proximal end and a distal end, and may define one or more tubular passages or “lumens” extending through part or all of the catheter shaft. Such lumens often have one or more openings, referred to as “ports,” or a lumen may have a closed lumen.

When a lumen is adapted to slidingly receive a guidewire, it is referred to as a “guidewire lumen,” and it will generally have a proximal and distal “guidewire port.” The distal guidewire port is often at or near the catheter shaft distal end.

A hub is often affixed to the catheter shaft proximal end. The hub may serve a variety of functions, including providing a handle for manipulating the catheter, and/or defining proximal port(s) communicating with lumen(s) defined by the catheter shaft. When the catheter has a guidewire lumen, a proximal guidewire port may be located at some point along the sidewall of the catheter shaft, or a hub may define the proximal guidewire port.

A guidewire has a flexible wire-like structure extending from a proximal end to a distal end. The guidewire will usually be of a size selected to fit into and slide within a corresponding guidewire lumen of a catheter.

Catheter balloons represent another possible example of this invention. Since rigid-rod paraphenylene polymer materials can be extruded into thin-wall tubes, biaxially orienting the tube into a high strength balloon is feasible. Since rigid-rod paraphenylene boasts tensile strength of up to 30 ksi, balloons prepared from this material are very thin and strong. For example, balloons of rigid-rod paraphenylene may have nominal inflation pressures well in excess of 25 atmospheres.

Other possible examples of this invention include guidewires, hypotubes, and marker bands. Because the strength of rigid-rod paraphenylene puts this property in the range of metals, and its ability to extrude, injection mold and solvent-cast, replacement of metal components in catheter systems is possible. This feature is particularly beneficial for MR (magnetic resonance) compatible catheter systems. In the case of marker bands, rigid-rod paraphenylene can be doped with appropriate radiopaque material and then extruded to create marker bands with low profile.

The terms “tube” and “tubular” are used in their broadest sense, to encompass any structure arranged at a radial distance around a longitudinal axis. Accordingly, the terms “tube” and “tubular” include any structure that (i) is cylindrical or not, such as for example an elliptical or polygonal cross-section, or any other regular or irregular cross-section; (ii) has a different or changing cross-section along its length; (iii) is arranged around a straight, curving, bent or discontinuous longitudinal axis; (iv) has an imperforate surface, or a periodic or other perforate, irregular or gapped surface or cross-section; (v) is spaced uniformly or irregularly, including being spaced varying radial distances from the longitudinal axis; or (vi) has any desired combination of length or cross-sectional size.

Any suitable additional material may also be used to make catheters and hubs as described, including polymers and other materials suitable for use with medical devices.

It is of course possible to build various kinds and designs of catheters according to the present invention, by various techniques and of various materials, to obtain the desired features. It should be noted that the present invention also relates to methods for making and using medical devices, during or in preparation for medical treatment of a patient.

These and various other objects, advantages and features of the invention will become apparent from the following description and claims, when considered in conjunction with the appended drawings. The invention will be explained in greater detail below with reference to the attached drawings of a number of examples of embodiments of the present invention.

### BRIEF DESCRIPTION OF THE DRAWINGS

FIG. 1 is a perspective view of an over-the-wire balloon catheter;

FIG. 2 is a perspective view of a catheter;

FIG. 3 is a perspective view of a rapid-exchange balloon catheter; and

FIG. 4 is a partial side elevation and partial cross-section view of a guidewire.

### DETAILED DESCRIPTION OF THE PREFERRED EMBODIMENTS

The following description of the preferred embodiments of the present invention is merely illustrative in nature, and as such it does not limit in any way the present invention, its application, or uses. Numerous modifications may be made by those skilled in the art without departing from the true spirit and scope of the invention.

The drawings depict medical devices along the lines of the present invention, which include one or more components made of rigid-rod paraphenylene.

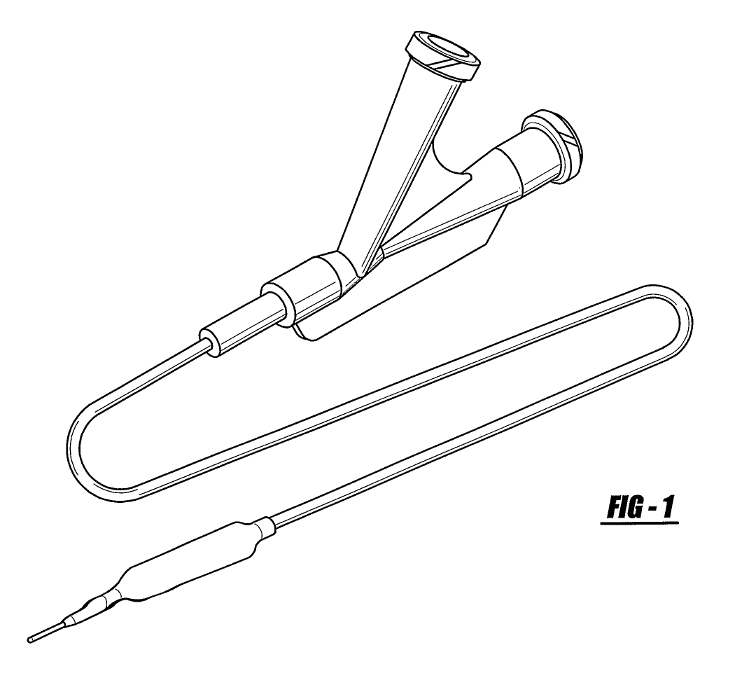

In FIG. 1, a balloon catheter has a flexible shaft extending between proximal and distal ends, a balloon affixed to the shaft near the distal end, a hub affixed to the proximal end, and a strain relief positioned at the transition. The hub defines an inflation port communicating with an inflation lumen so the balloon can be inflated and deflated, and a proximal guidewire port allowing a guidewire to be introduced through a guidewire lumen. Because the proximal guidewire port is defined by the hub, the balloon catheter shown in FIG. 1 has an “over-the-wire” arrangement.

Any or all of the components of the balloon catheter shown in FIG. 1 may be made using the rigid-rod paraphenylene of the present invention. Due to the physical properties of rigid-rod paraphenylene, including very high tensile strength, no additional reinforcing members such as stiffening wires, coils, or braids are needed. Also, the self-reinforcing nature of rigid-rod paraphenylene allows for thinner wall extrusions without sacrificing strength, which translates into catheter deliverability, pushability and steerability.

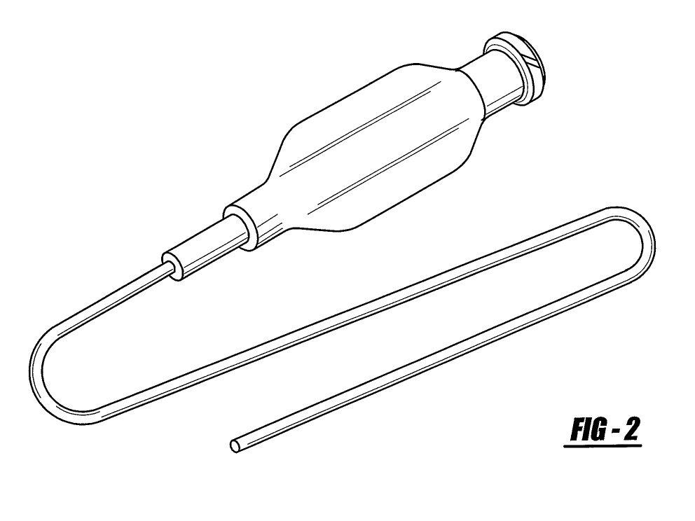

FIG. 2 shows for example a diagnostic catheter or a guiding catheter having a flexible tubular shaft extending between proximal and distal ends, a hub affixed to the proximal end, and a strain relief positioned at the transition.

Again, any or all of the components of the diagnostic catheter shown in FIG. 2 may be made using the rigid-rod paraphenylene of the present invention.

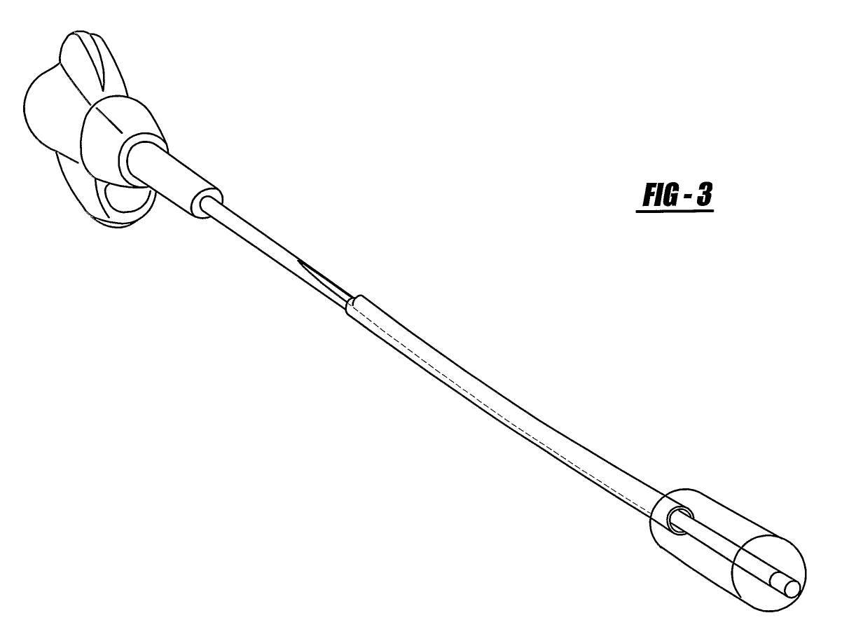

FIG. 3 shows another balloon catheter, similar to that of FIG. 1, except that the proximal guidewire port is located at an intermediate location on the catheter shaft, between the hub and the balloon. The balloon catheter shown in FIG. 3 therefore has a “rapid-exchange” arrangement.

Likewise, any or all of the components of the diagnostic catheter shown in FIG. 2 may be made using the rigid-rod paraphenylene of the present invention.

Examples of additional types of catheters having rigid-rod paraphenylene materials include stent delivery system catheters, injection catheters, gene therapy catheters, electrophysiology catheters, therapeutic drug delivery catheters, ultrasound catheters, laser angioplasty catheters, etc.

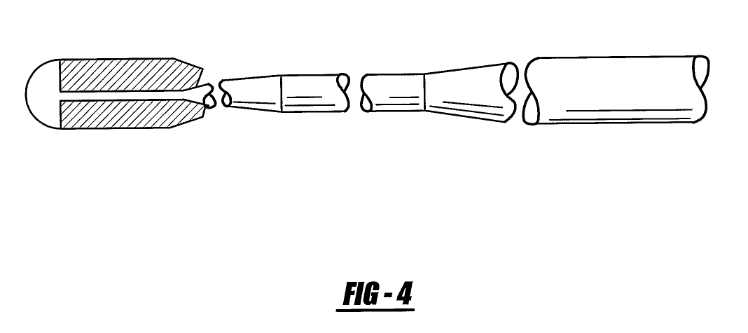

FIG. 4 shows another example medical device according to the principles of the present invention, which is a flexible guidewire. The example guidewire shown in FIG. 4 has cylindrical and tapering portions, a narrow distal section having a surrounding coil, and a distal tip. As with all of the medical devices of the present invention, any or all of the components of the guidewire may be made using the rigid-rod paraphenylene of the present invention.

Medical devices according to the principles of the present invention may be made of any suitable materials in addition to rigid-rod paraphenylene, using a variety of methods. Various other polymers have desired characteristics of strength, resilience, flexibility, biocompatibility and endurance. For example, various polymers may be used, such as nylons and other polyamides, as well as polyimides, polycarbonate, polypropelene, ABS, and polyethylenes.

It should be understood that an unlimited number of configurations for the present invention could be realized. The foregoing discussion describes merely exemplary embodiments illustrating the principles of the present invention, the scope of which is recited in the following claims. Those skilled in the art will readily recognize from the description, claims, and drawings that numerous changes and modifications can be made without departing from the spirit and scope of the invention.

What is claimed is:

1. A medical device for therapeutically treating a patient by advancing along a body passage to a desired site for treatment, comprising:
a flexible polymer component made only of rigid-rod paraphenylene, wherein the component has sufficient strength and flexibility that the component has no reinforcement.

2. The medical device of claim 1, wherein the medical device is a catheter, and the component is a catheter shaft defining a lumen.

3. The medical device of claim 2, further comprising a balloon affixed to a distal end of the catheter shaft.

4. The medical device of claim 3, wherein the balloon is made of a thin film of rigid-rod paraphenylene.

5. The medical device of claim 1, wherein the medical device is a guidewire.

6. The medical device of claim 1, wherein the type of medical device is selected among the group of: balloon catheters, diagnostic catheters, guiding catheters, stent delivery system catheters, injection catheters, gene therapy catheters, electrophysiology catheters, therapeutic drug delivery catheters, ultrasound catheters, and laser angioplasty catheters.

7. A balloon catheter, comprising:
a catheter shaft having a proximal and distal end, defining an inflation lumen;
a polymer balloon affixed to the catheter shaft near the distal end, the inflation lumen communicating with an interior of the balloon;
at least a portion of the balloon catheter being made of rigid-rod paraphenylene.

8. The balloon catheter of claim 7, wherein the balloon is made of said rigid-rod paraphenylene.

9. The balloon catheter of claim 7, wherein at least a portion of the catheter shaft is formed said rigid-rod paraphenylene.

10. The balloon catheter of claim 7, wherein the balloon material is substantially inelastic.

11. The balloon catheter of claim 7, wherein the balloon is an angioplasty balloon.

12. The balloon catheter of claim 7, further comprising a stent crimped around the balloon, such that inflation of the balloon will expand and deploy the stent.

13. The balloon catheter of claim 7, wherein the balloon catheter has a nominal inflation pressure equal to or greater than 25 atmospheres.

14. A catheter, comprising a tubular shaft member, at least a portion of the shaft member being made of rigid-rod paraphenylene.

15. The catheter of claim 13, at least a portion of the shaft member being made of a blend of rigid-rod paraphenylene and another polymer.
